(19) United States
(12) Patent Application Publication
(10) Pub. No.: US 2023/0400452 A1
(43) Pub. Date: Dec. 14, 2023

(54) Sample Analysis Chip and Sample Analysis Method Using Sample Analysis Chip

(71) Applicant: TOTAL BIOSENSE INC., Namyangju-si (KR)

(72) Inventors: Tae Seok SEO, Suwon-si (KR); Van Hau NGUYEN, Yongin-si (KR)

(73) Assignee: TOTAL BIOSENSE INC., Namyangju-si (KR)

(21) Appl. No.: 18/111,866

(22) Filed: Feb. 20, 2023

Related U.S. Application Data
(63) Continuation of application No. PCT/KR2021/011235, filed on Aug. 23, 2021.

(30) Foreign Application Priority Data
Aug. 21, 2020 (KR) 10-2020-0105566
Sep. 21, 2020 (KR) 10-2020-0121609
Sep. 21, 2020 (KR) 10-2020-0121610

(51) Int. Cl. G01N 33/53; G01N 33/543; G01N 21/64

(52) U.S. Cl. CPC G01N 33/5304; G01N 21/6428; G01N 33/54386

(57) ABSTRACT

The present invention provides a chip for sample assay and a sample assay method using same, the chip being capable of assaying a target analyte included in a sample, even with a trace amount of the sample, due to improved efficiency in capture of the target analyte included in the sample.

### TECHNICAL HELD

The present invention relates to a sample analysis chip and a sample analysis method using the sample analysis chip.

### BACKGROUND ART

Gene diagnosis is essential for diagnosis of bacteria or viral diseases.

The gene diagnosis consists of four steps including a sample collection step, a sample: pretreatment step (DNA or RNA extraction), a gene amplification step, and a detection step.

However, in the related art, since each step is performed by separate equipment or devices, a high-priced analysis device and a large amount of a sample are required, a large amount of time is required for analysis, the possibility of contamination of a sample during diagnosis is high, and rapid diagnosis at a site is difficult.

In order to solve this problem, an integrated gene analysis apparatus using a microchip based on microfluidic has been developed. Because the laboratory environment is implemented on one chip, it is called a Lab on a Chip.

However, conventional integrated gene analysis apparatuses also have problems such as high-priced manufacturing costs due to complex chip structures, metal electrode patterning, and silicon/glass substrate-based structures, complexity of operation due to the necessity of external inflow pumps and multiple tube system, low reproducibility of a highly integrated chip-driving apparatus, and difficulties in on-site diagnosis due to the lack of automatic operation and limitation of miniaturization. Thus, improvements are required.

In the related art, Korean Patent Laid-Open No. 10-2020-0064466, which is disclosed by the inventor of the present invention, has been used. The prior art relates to a sample analysis chip and uses a principle in which a material to be analyzed included in a sample is captured by beads provided in a capture passage while passing through a zigzag-shaped capture passage when a sample is injected. However, since the space between the beads is not uniform, there is a problem in that the efficiency of extracting the material to be analyzed from the sample is very deteriorated. The prior art discloses an oil loading unit and the liquid oil stored in the oil loading unit is injected into the front end of the reaction chamber. However, because the oil accommodated in the oil loading unit is in a liquid state, the oil leaks during the rotation of the sample analysis chip even when the outlet of the oil loading unit is sealed by a sealing unit such as paraffin wax. So, there was a problem of contamination by oil even before the sample was introduced into the reaction chamber.

(Patent Document 1) Korean Patent No. 10-1965963 (13 Aug. 2019)

(Patent Document 2) Korean Patent No. 10-1986464 (5 Jun. 2019)

Patent Document 3) Korean Patent No. 10-2076809 (17 Feb. 2020)

(Patent Document 4) Korean Patent Laid-Open No. 10-2020-0064466 (8 Jun. 2020)

(Patent Document 5) Korean Patent Laid-Open No. 10-2018-0128054 (30 Nov. 2018)

(Patent Document 6) Korean Patent No. 10-1091906 (2 Dec. 2011)

(Patent Document 7) Korean Patent Laid-Open No. 10-2009-0112560 (28 Oct. 2009)

### DETAILED DESCRIPTION OF THE INVENTION

### Technical Problem

t is an object of the present invention to provide an integrated micro device capable of implementing all gene diagnosis in one chip.

An object of the present invention is to provide a chip having improved capture efficiency of a material to be analyzed included in a sample when compared to a conventional sample analysis chip.

In addition, the purpose of the present invention is to provide a chip for solving a problem of a conventional sample analysis chip, that is a liquid wax is discharged before the pretreated sample is injected into the reaction chamber, thereby reducing the accuracy of analysis, by providing a wax storage unit in which wax in a solid state is stored and the wax is not discharged to the reaction chamber before the wax storage unit is heated.

Another object of the present invention is to provide an analysis device in which pretreatment, gene amplification, and analysis processes of a sample analysis chip are performed automatically in one analysis device.

### Problem Solving Means

According to an embodiment of the present invention, a sample analysis chip is provided, which may comprise
a sample storage unit 110;
a capture passage 120 communicating with the sample storage unit 110, having a membrane F configured to capture an analysis target material contained in the sample injected into the sample storage unit 110, and positioned radially outward than the sample storage unit 110;
a washing liquid storage unit 130 positioned radially inward than the sample storage unit 110 and communicating with the capture passage 120;
a cocktail storage unit 150 positioned radially inward than the sample storage unit 110 and into which a cocktail for detection of the analysis target material is introduced;
an eluent storage unit 160 positioned radially inward than the sample storage unit 110, communicating with the capture passage 120, and into which an eluent for separating the analysis target material captured in the capture passage 120 is introduced;
a connection chamber 170 positioned radially outward than the capture passage 120 and the cocktail storage unit 150 and communicating with the outlet of the capture passage 120 and the cocktail storage unit 150;
a collection chamber 172 positioned radially outward than the connection chamber 170 and communicating with the connection chamber 170, and into which an eluate comprising the analysis target material and the cocktail are introduced when the sample analysis chip is rotated to analyze the sample;
a reaction chamber 180 positioned radially outward than the collection chamber 172 and communicating with the collection chamber 172; and
a wax storage unit 190 storing a solid wax therein positioned radially inward than the reaction chamber 180 and communicating with the reaction chamber 180.

According to an embodiment of the present invention, the sample analysis chip may comprise an input channel 174 positioned radially outward than the collection chamber 172, positioned radially inward than the reaction chamber 180, communicating with the collection chamber 172 and the reaction chamber 180, and into which a mixture of an eluate containing the analysis target material and the cocktail is introduced; and
a connection channel 175 connecting the input channel 174 and the reaction chamber 180.

According to an embodiment of the present invention, the sample analysis chip may have a plurality of reaction chambers 180, the plurality of reaction chambers 180 may be formed along the circumferential direction of the sample analysis chip, and the length of the connection channel 175 may be shorter as the distance from the collection chamber 172 increases.

According to an embodiment of the present invention, the sample analysis chip may comprise a disk 10;
a first film layer 20 formed on an upper portion of the disk 10; and
a second film layer 30 formed on an under portion of the disk 10,
wherein the sample storage unit 110, the capture passage 120, the washing liquid storage unit 130, the cocktail storage unit 150, the eluent storage unit 160, the connection chamber 170, the collection chamber 172, the reaction chamber 180, and the wax storage unit 190 may be provided in a groove shape in the disk 10.

According to an embodiment of the present invention, the sample analysis chip may further comprise
a first delay chamber 142 provided between the washing liquid storage unit 130 and the capture passage 120, and
a second delay chamber 164 provided between the eluent storage unit 160 and the capture passage 120,
wherein the sample storage unit 110, the first delay chamber 142, and the second delay chamber 164 may be provided in a groove shape on an upper portion of the disk 10, the connection chamber 170 may be provided in a groove shape on an under portion of the disk 10, and
the capture passage 120 may be provided in a groove shape extending in an upward direction and a downward direction of the disk 10 so that the sample storage unit 110, the first delay chamber 142, and the second delay chamber 164 are communicating with the connection chamber 170.

According to an embodiment of the present invention, the membrane F may be a glass filter in which a plurality of silica beads is provided in a membrane form, and the glass filter made of three layers of the silica beads may be installed on the capture passage 120.

According to an embodiment of the present invention, injection holes 111, 131, 151, and 161 for solution injection may be formed in each of the sample storage unit 110, the washing liquid storage unit 130, the cocktail storage unit 150, and the eluent storage unit 160.

According to an embodiment of the present invention, the sample analysis chip may further comprise a cartridge 200 having each inlet and each solution storage unit, wherein the each inlet communicates with the each solution storage unit, and the each inlet is connected to the each injection holes 111, 131, 151, and 161, injecting the sample into the sample storage unit 110, injecting the washing liquid into the washing liquid storage unit 130, injecting the cocktail into the cocktail storage unit 150, and injecting the eluent into the eluent storage unit 160.

According to an embodiment of the present invention ; the sample analysis chip may further comprise
an inflow channel 141; and
a delay channel 143,
wherein the inflow channel 141 may be disposed between the washing liquid storage unit 130 and the first delay chamber 142 to allow the washing liquid injected into the washing liquid storage unit 130 to be introduced into the first delay chamber 142 when the sample analysis chip is rotated to analyze the sample,
the inflow channel 141 may include a first passage 141a extending away from the first delay chamber 142 in a first circumferential direction and a second passage 141b extending toward the first delay chamber 142 in a second circumferential direction opposite to the first circumferential direction at an end of the first passage 141a,
the delay channel 143 may be disposed between the first delay chamber 142 and the capture passage 120 to allow the washing liquid is discharged into the capture passage 120 when the washing liquid injected into the first delay chamber 142 is greater than a predetermined amount,
the delay channel 143 may include a first delay passage 143a extending radially inward at the outlet of the first delay chamber 142 and a second delay passage 143b extending radially outward than an end of the first delay passage 143a.

According to an embodiment of the present invention, a thickness of the sample analysis chip where the reaction chamber 180 is provided may be thinner than that of other portions of the sample analysis chip.

According to an embodiment of the present invention, an apparatus may be provided. In the apparatus ; a sample analysis chip 100 may be installed, the sample analysis chip 100 may comprise:
a sample storage unit 110;

a pretreatment unit located radially outward than the sample storage unit 110 and the sample introduced into the sample storage unit 110 is pretreated; and
a reaction chamber 180 located radially outward than the pretreatment unit and into which the sample pretreated by the pretreatment unit is introduced,
the apparatus analyzes a material introduced into the reaction chamber 180 by amplifying the material in the reaction chamber 180, the apparatus may comprise:
   a chip installation part 320 on which the sample analysis chip 100 is seated, which includes a motor 322 that rotates the sample analysis chip 100;
   a heating part 330 configured to be movable closer to or away from the reaction chamber 180 in alignment up and down relative to the reaction chamber 180, and including a first heater 331 for heating the reaction chamber 180; and
   a sensor part 350 in alignment up and down relative to the reaction chamber 180 and capable of detecting fluorescence generated in the reaction chamber 180.

According to an embodiment of the present invention, the sample analysis chip 100 of the apparatus may further include a wax storage unit 190 storing a solid wax therein, positioned radially inward than the reaction chamber 180, and communicating with the reaction chamber 180,
and the heating part 330 may further include a second heater 332 spaced apart from the first heater 331 in alignment up and down relative to the wax storage unit 190 and heating the wax storage unit 190.

According to an embodiment of the present invention, the heating part 330 of the apparatus may further include a Peltier element 333 connected to the first heater 331.

According to an embodiment of the present invention, the heating part 330 of the apparatus may be installed on an upper side and a lower side of the reaction chamber 180, respectively.

According to an embodiment of the present invention, the apparatus may be provided with a plurality of reaction chambers 180, and the plurality of reaction chambers 180 may be formed along a circumferential direction of the sample analysis chip 100, and the first heater 331 may extend along the circumferential direction.

According to an embodiment of the present invention, the area of the first heater 331 of the apparatus may be configured to cover at least all of the plurality of reaction chambers 180, and the area of the second heater 332 of the apparatus may be configured to at least cover the wax chamber 190.

According to an embodiment of the present invention, a thickness of a portion where the reaction chamber 180 of the sample analysis chip 100 is formed may be thinner than that of the other portions.

According to an embodiment of the present invention, the apparatus may further comprise an operation processor part 360 configured to determine whether a material to be analyzed is present in the sample injected into the sample storage unit 110 or whether the sample is infected with a disease, based on the fluorescence intensity detected by the sensor part 350.

### Effect of the Invention

According to the present invention, all gene diagnosis can be implemented in one chip.

In addition, when compared to a conventional sample analysis chip, the capture efficiency of the analysis target material included in the sample is improved.

In addition, since the wax in the solid state is stored in the wax storage unit and the wax is not discharged to the outside before the wax storage unit is heated, the problem of a conventional sample analysis chip, that is, the wax is discharged before the pretreated sample is injected into the reaction chamber, thereby the accuracy of analysis is lowered, is solved.

In addition, pretreatment, gene amplification, and analysis of the sample analysis chip may be performed automatically in one analysis device.

In addition, according to the present invention, it is possible to immediately diagnose on site whether a pathogen having a large social wavelength such as influenza virus, avian influenza virus, MERS virus, Zika virus, and foot-and-mouth disease occurs.

According to the present invention, it is possible to block virus infection in advance, and to reduce loss of human life and economic loss.

A gene diagnosis that can be performed only in a large medical institution, such as a. university hospital, can be quickly performed in a small medical institution and a health care center.

In addition, it is possible to diagnose on site viruses/bacteria that infects food such as food poisoning bacteria, and thus it is possible to continuously monitor widespread problems that occur when meal is served in a kindergarten, an elementary school, a middle school, and a high school.

### BRIEF DESCRIPTION OF THE DRAWINGS

FIG. 1 is a view for explaining a sample analysis chip according to the present invention.

FIG. 2 is a view illustrating a unit process part of the sample analysis chip of FIG. 1.

FIG. 3 is a cross-sectional view illustrating a capture passage and a configuration connected to the capture passage.

FIG. 4 is a view illustrating a cartridge connected to the sample analysis chip of FIG. 1 to inject a solution.

FIG. 5 is a diagram of an analysis apparatus in which a sample analysis chip according to the present invention is installed to perform pretreatment, gene amplification, and analysis of a sample injected into the sample analysis chip.

FIG. 6 is a diagram illustrating an internal configuration of the analysis apparatus of FIG. 5.

FIG. 7 is a view illustrating a heating part for heating a reaction chamber and a wax storage unit of a sample analysis chip.

FIG. 8 is a view illustrating a second heater configuration of the heating part of FIG. 7.

FIG. 9 is a view illustrating a state in which a heating part is aligned up and down relative to a reaction chamber and a wax storage unit.

FIG. 10 is a schematic view illustrating a state in which heating is performed by a heating part aligned up and down relative to a reaction chamber and a wax storage unit of a sample analysis chip.

FIG. 11 is a schematic view illustrating a state in which a driving parts of a sample analysis chip is aligned up and down relative to a reaction chamber to detect an optical signal in the driving parts.

FIGS. 12A to 12L are views for explaining a pretreatment process and a gene amplification process of a sample put into a sample analysis chip using a sample analysis chip according to the present invention.

FIG. 13 is a diagram illustrating a result of verification experiment 1.

FIGS. 14 and 15 are diagrams of results according to verification experiment 2.

FIGS. 16 to 20 are diagrams of results according to verification experiment 3.

FIGS. 21 and 22 are diagrams of results according to verification experiment 4.

FIG. 23 is a diagram of a conventional sample analysis chip.

Form for Implementation of the Invention

Hereinafter, the term “gene amplification” means that a gene to be analyzed is amplified. As an example of gene amplification, there may be a polymerase chain reaction (PCR), a real-time PCR, and an isothermal amplification reaction. Any reaction to amplify a gene to be analyzed is included therein without limitation.

### SAMPLE ANALYSIS CHIP

Hereinafter, the sample analysis chip 100 according to the present invention will be described in detail with reference to the accompanying drawings.

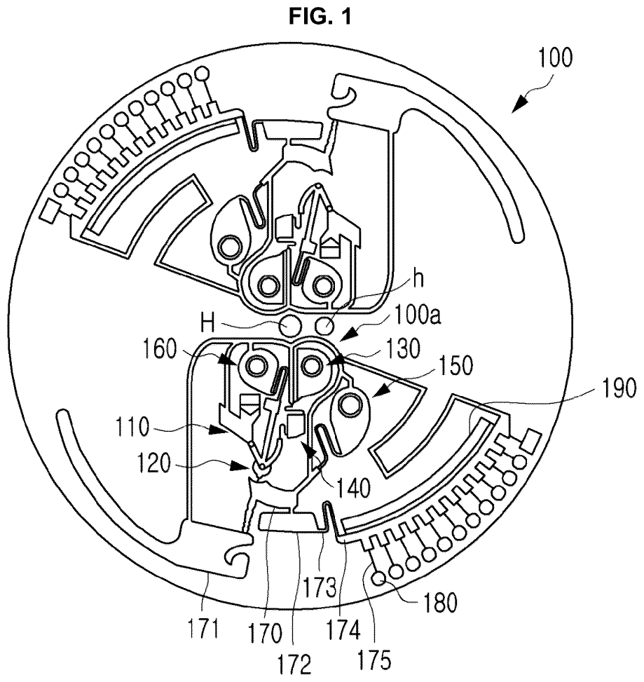

Referring to FIG. 1, the sample analysis chip 100 may have a circular plate shape, and a first through-hole H may be formed at a center of the chip 100. The first through-hole H is coupled to the chip analysis apparatus 300 for sample analysis, which will be described later, and is a part corresponding to a rotary shaft when the sample analysis chip is rotated. A second through-hole h is formed outside the first through-hole H in the radial direction, and a vibration prevention part 400, which will be described later, is coupled to the second through-hole h such that when the sample analysis chip 100 is rotated at a high speed, the problem that the sample analysis chip 100 vibrates or the solution is discharged from the cartridge 200 to the outside is resolved. This will be described in detail below.

The sample analysis chip 100 is composed of three layers. That is, an upper surface and a lower surface of a circular plate-shaped PMMA having a predetermined thickness, for example, a thickness of 3 mm, may be processed using a CNC milling machine to form a predetermined pattern in the form of a groove, and then the PSA film may be adhered to the upper surface and the lower surface.

That is, the sample analysis chip 100 includes a disk 10, a first film layer 20 adhered to an upper surface of the disk 10, and a second film layer 30 adhered to a lower surface of the disk 10. In addition, the components to be described below are patterned in a groove shape on the upper surface or the lower surface of the disk 10, and the first film layer 20 covers the upper portion of each patterned configuration, and the second film layer 30 covers the lower portion of each patterned configuration.

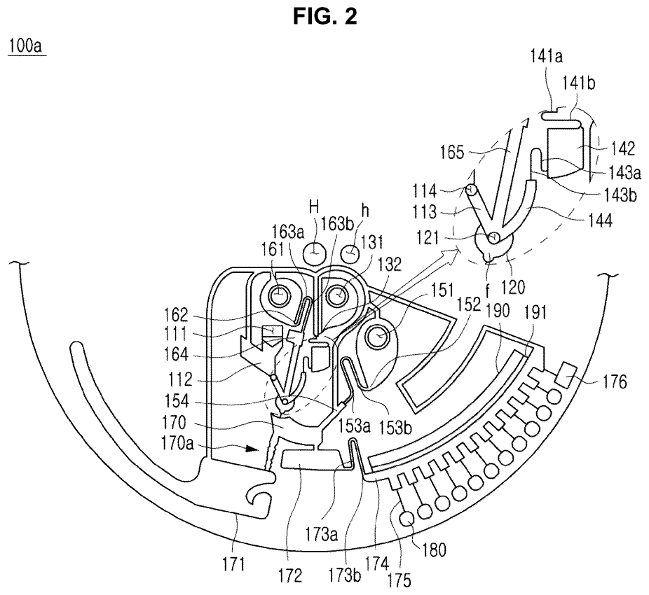

The sample analysis chip 100 according to the present invention includes a plurality of unit process parts 100a sequentially arranged in a circumferential direction. FIG. 1 illustrates an embodiment in which two unit process parts are formed on a sample analysis chip, and FIG. 2 is a view for specifically explaining one unit process part.

The unit process part 100a is formed in a pattern on the disk 10, and includes a sample storage unit 110, a capture passage 120, a washing liquid storage unit 130, a delay unit 140, a cocktail storage unit 150, an eluent storage unit 160, a connection chamber 170, a waste liquid chamber 171, a collection chamber 172, a reaction chamber 180, and a wax chamber 190. Inlets 111, 131, 151, and 161 are formed in the sample storage unit 110, the washing liquid storage unit 130, the cocktail storage unit 150, and the eluent storage unit 160, respectively, and the cartridge 200 is mounted on the sample analysis chip 100 according to the present invention to inject each solution.

That is, the sample inlet 211, the washing liquid inlet 231, the cocktail inlet 251, and the eluent inlet 261 are formed in the cartridge 200, respectively, and spaces 210, 230, 250, and 260 communicating with the inlets 211, 231, 251, and 261 are formed in the cartridge 200, so that each solution is stored in the space, and when the sample analysis chip 100 is rotated, the solutions stored in the cartridge 200 may be injected into the sample storage unit 110, the washing liquid storage unit 130, the cocktail storage unit 150, and the eluent storage unit 160.

The sample storage unit 110 is positioned more radially inward than the capture passage 120 to load the sample injected from the cartridge 200 into the capture passage 120.

During the rotation of the sample analysis chip 100, the loaded sample flows to the waste liquid chamber 171 through the capture passage 120 and the connection chamber 170 by centrifugal force. More specifically, the sample introduced into the sample injection inlet 111 from the cartridge 200 sequentially passes through the input portion 112 and the sample input channel 113 and is introduced into the capture passage 120. Portions 114 and 121 not covered by the first film layer 20 exist at a connection point between the input portion 112 and the sample input channel 113, and a connection point between the sample input channel 113 and the capture passage 120, and in other words, external air is introduced through the portions 114 and 121 to allow the sample injected into the sample storage unit 110 to be more quickly moved to the capture passage 120. Therefore, the washing liquid injected into the washing liquid storage unit 130 may be prevented from reaching the capture passage 120 before the sample reaches the capture passage 120, or the washing liquid injected into the washing liquid storage unit 130 may be prevented from reaching the capture passage 120 before all the samples pass through the capture passage 120.

The capture passage 120 captures an analysis target material from a sample loaded through the sample storage unit 110. An inlet 122 and an outlet 123 are provided at both ends of the capture passage 120, respectively. The inlet 122 of the capture passage 120 communicates with the sample: storage unit 110, the washing liquid storage unit 130, and the eluent storage unit 160 located radially inward than the capture passage 120, and the outlet 123 of the capture passage 120 communicates with the connection chamber 170 located radially outward than the capture passage 120. Accordingly, the sample, the washing liquid, and the eluent may flow into the connection chamber 170 according to the rotation of the sample analysis chip 100.

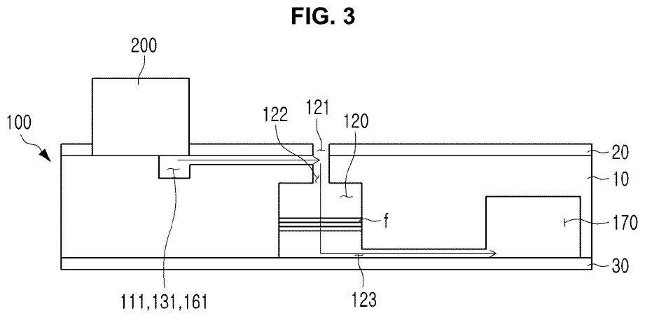

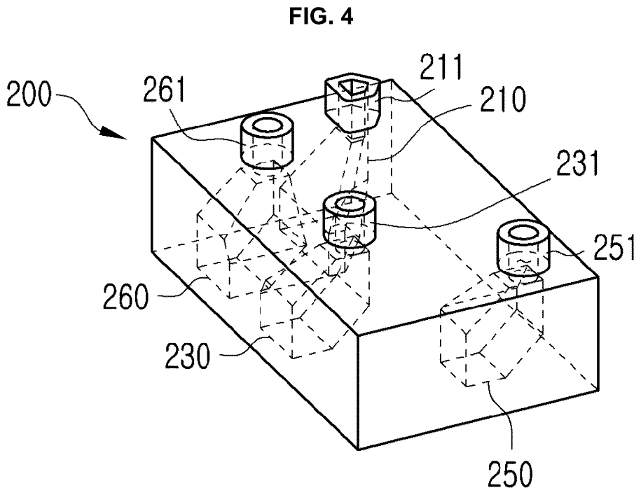

The configurations of the sample storage unit 110, the washing liquid storage unit 130, and the eluent storage unit 160 communicating with the inlet 122 of the capture passage 120 are provided in a groove shape on the upper surface of the disk 10, and configurations of the connection chamber 170, the waste liquid chamber 171, the collection chamber 172, the input channel 174, the reaction chamber 180, and the wax storage unit 190 communicating with the outlet 122 of the capture passage 120 are provided in a groove shape on the lower surface of the disk 10. The capture passage 120 is provided in the form of a groove on the upper surface and lower surface of the disk 10. For the communication of configurations of the capture passage 120 on the upper surface and the lower surface of the disk 10, the capture passage 120 is provided in the form of a groove extending in the vertical direction of the disk 10 (see FIG. 3).

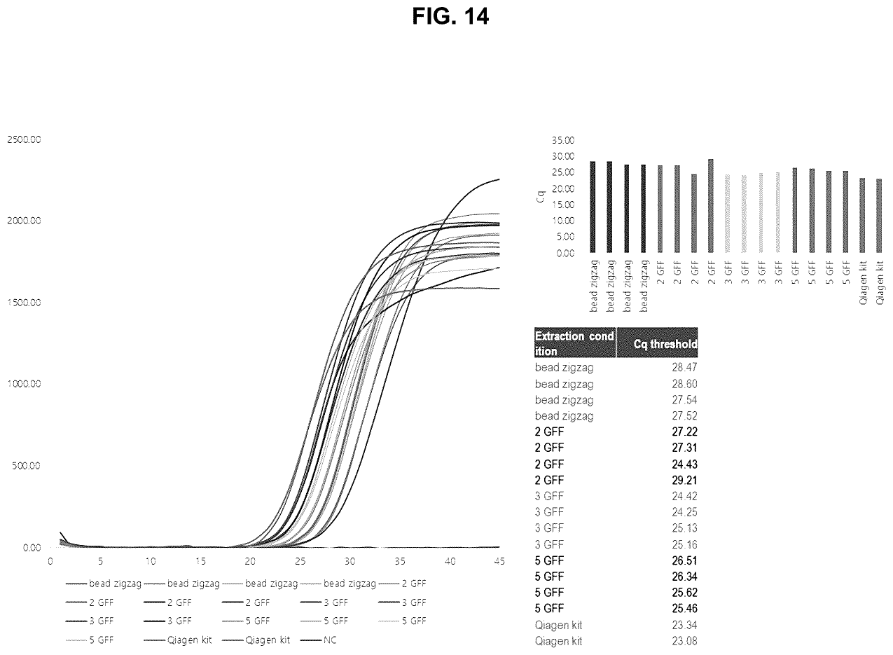

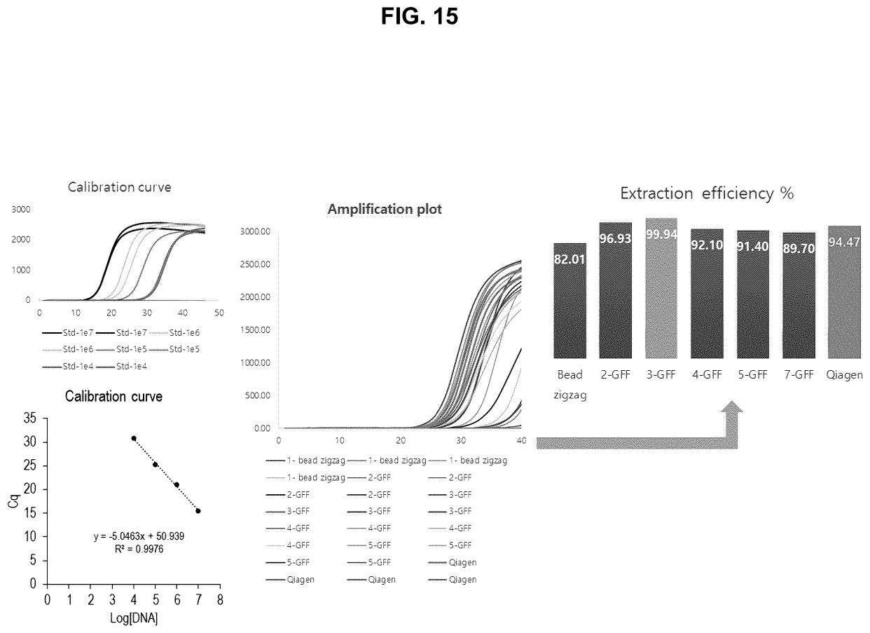

A membrane F for capturing an analysis target material is provided on the capture passage 120. The membrane F according to the present invention may be a glass filter, and the glass filter may have a form in which a plurality of silica beads is provided in a membrane form. Specifically, one or more glass filters may be installed on the capture passage 120. More preferably, a glass filter composed of three layers of a plurality of silica beads is installed on the capture passage 120 to capture the material to be analyzed with high efficiency from the sample (see FIGS. 14 and 15). The silica bead of the glass filter has a negative charge, and when the glass filter is formed to include a salt such as guanidine salt having a positive charge, the glass filter can capture an analysis target material such as DNA genome through the binding of the silica bead-guanidine salt-analysis target material having negative charge (DNA etc.).

The washing liquid storage unit 130 may be in the form of a chamber, and may store a washing liquid for cleaning (or removing) the remaining material other than the material to be analyzed captured by the capture passage 120. The washing liquid storage unit 130 is positioned radially inward than the capture passage 120 and is connected to the inlet 122 of the capture passage 120. Accordingly, the washing liquid stored in the washing liquid storage unit 130 flows to the capture passage 120 according to the rotation of the sample analysis chip 100, and thus the remaining material other than the analysis target material captured in the capture passage 120 may be washed (removed).

A delay unit 140 may be provided between the washing liquid storage unit 130 and the capture passage 120. The delay unit 140 is configured to delay the reach of the washing liquid from the washing liquid storage unit 130 to the capture passage 120 later than the sample.

The delay unit 140 includes an inflow channel 141, a first delay chamber 142, and a delay channel 143.

The inflow channel 141 connects the washing liquid storage unit 130 to the first delay chamber 142 and allows the washing liquid injected into the washing liquid storage unit 130 to flow into the first delay chamber 142. The inflow channel 141 includes a first passage 141a extending from the outlet 132 of the washing liquid storage unit 130 in a first circumferential direction away from the first delay chamber 142 and a second passage 141b extending from the first passage 141a in a second circumferential direction opposite the first circumferential direction toward the first delay chamber 142.

The first delay chamber 142 stores a washing liquid directed from the washing liquid storage unit 130 to the capture passage 120.

The delay channel 143 is provided between the first delay chamber 142 and the capture passage 120 to delay passage of the washing liquid. To this end, the delay channel 143 includes a first delay passage 143a extending radially inward and a second delay passage 143b connected to the first delay passage 143a and extending radially outward. Accordingly, since the washing liquid is not injected into the capture passage 120 through the delay channel 143 before the washing liquid stored in the first delay chamber 142 reaches a preset amount, the washing liquid reaches the capture passage 120 later than the sample. Therefore, it is possible to prevent the washing liquid from flowing into the capture passage 120 before the material to be analyzed is captured.

A cocktail is put into the cocktail storage unit 150. Here, the cocktail may include a material required for PCR or RT-PCR such as a gene amplification enzyme (for example, a DNA polymerase) or MgCl₂ salt.

The cocktail storage unit 150 is connected to the connection chamber 170 by the cocktail introduction channel 153.

The cocktail introduction channel 153 includes a third passage 153a extending radially inward and a fourth passage 153b connected to the third passage 153a and extending radially outward. Therefore, when the sample analysis chip 100 rotates, the cocktail is not introduced into the connection chamber 170, and when the sample analysis chip 100 is stopped, the cocktail may be introduced into the connection chamber 170. Therefore, after all of the sample and the washing liquid are introduced into the capture passage 120, the cocktail may be sequentially introduced into the connection chamber 170. The above-described cocktail introduction channel 153 may be a hydrophilic coated structure.

The eluent storage unit 160 may inject an eluent for separating the analysis target material captured in the capture passage 120. The eluent storage unit 160 is positioned radially inward than the capture passage 120 and communicates with the capture passage 120. Accordingly, as the eluent stored in the eluent storage unit 160 flows to the capture passage 120 according to the rotation of the sample analysis chip 100, the analysis target material captured in the capture passage 120 may be separated from the membrane F.

The eluent storage unit 160 is connected to the capture passage 120 through an eluent introduction channel 163.

The eluent introduction channel 163 includes a fifth passage 163a extending radially inward and a sixth passage 163b connected to the fifth passage 163a and extending radially outward. Therefore, when the sample analysis chip 100 rotates, the eluent may not flow into the capture passage 120, and when the sample analysis chip 100 stops, the eluent may flow into the capture passage 120. Therefore, after all of the sample and the washing liquid are introduced into the capture passage 120, the eluent may be sequentially introduced into the capture passage 120. The above-described eluent introduction channel 163 may be a hydrophilic coated structure.

The connection chamber 170 is located radially outward than the capture passage 120 and the cocktail storage unit 150 and is connected to the capture passage 120 and the cocktail storage unit 150.

A waste liquid chamber 171 and a collection chamber 172 are located radially outward than the connection chamber 170.

The sample and the washing liquid passing through the capture passage 120 flow to the waste liquid chamber 171, and the eluent passing through the capture passage 120 and the cocktail fed into the connection chamber 170 flow to the collection chamber 172.

The connection chamber 170 is connected to the waste liquid chamber 171 through a connection channel. At least one capillary valve 170a may be provided in the connection channel, and the capillary valve 170a may prevent the solution stored in the waste liquid chamber 171 from being introduced again into the connection chamber 170 through the connection channel. The above-described connection chamber 170 and the capillary valve 170a may have a hydrophobic coated structure.

The waste liquid chamber 171 stores the sample and the washing liquid that have passed through the capture passage 120. Unnecessary materials except for the material to be analyzed may be stored, and a super absorbent polymer absorbing the sample passing through the capture passage 120 may be attached to the inside thereof.

The collection chamber 172. stores and mixes the eluate containing the material to be analyzed and the cocktail.

A distribution portion is formed radially outward than the collection chamber 172. A mixed solution of the eluent and the master mix may be injected into the distribution portion.

A mixture introduction channel 173 is formed between the collection chamber 172 and the distribution portion, and the mixture introduction channel 173 includes a seventh passage 173a. extending radially inward and an eighth passage 173b connected to the seventh passage 173a and extending radially outward. Therefore, when the sample analysis chip 100 rotates, the mixture is not introduced into the distribution portion, and when the sample analysis chip 100 stops, the mixture may be introduced into the distribution portion. The above-described mixture introduction channel 173 may be a hydrophilic coated structure.

The distribution portion includes an input channel 174, a connection channel 175, and a reaction chamber 180.

The input channel 174 is connected to the collection chamber 172 through the mixture introduction channel 173, and distributes the mixture passing through the collection chamber 172 to at least one reaction chamber 180. To this end, the input channel 174 extends a predetermined length along the circumferential direction and has an aliquoting structure.

The input outlet formed in the input channel 174 is connected to the reaction chamber 180 and the connection channel 175. The connection channel 175 extends from each input outlet formed in the input channel 174 to the respective reaction chamber 180, and is formed to be narrower than the width of the input outlet formed in the input channel 174. Further, the length of each connection channel 175 may be shorter as it is located farther from the collection chamber 172 in the circumferential direction.

Each reaction chamber 180 is located radially outward than the connection channel 175 and corresponds to an input outlet formed in the input channel 174, respectively. The reaction chamber 180 receives the dispensed mixture through the input channel 174 and performs PCR or RT-PCR for the mixture dispensed according to the material to be analyzed. Different primers are stored in each reaction chamber 180 to detect the material to be analyzed included in each dispensed mixture. In addition, a material having a reference fluorescence intensity may be stored in one or more reaction chambers of the plurality of reaction chambers 180, and the information of the reference fluorescence intensity may be saved in advance in the chip analysis apparatus 300, which will be described later, to determine the reaction chamber indicating the reference fluorescence intensity during operation by the operation processor part 360 as a reference reaction chamber, thereby specifying the position of another reaction chamber.

The wax chamber 190 is positioned radially inward than the dispensing portion and is connected to the input channel 175 by a wax introduction channel 191. Wax in a solid state is stored in the wax chamber 190. The wax is used to prevent evaporation of the mixture after the mixture is dispensed into the reaction chamber 180. After the mixture is introduced into the reaction chamber 180, heat above a predetermined temperature may applied from the outside. Then, the melted wax (oil) may be introduced into the input channel 174 and the connection channel 175 to prevent evaporation of the mixture injected into the reaction chamber 180,

Since the wax chamber 190 stores wax in a solid state, if the wax chamber 190 is not heated, the wax is not injected into the input channel 175 even when the sample analysis chip 100 is rotated or stopped after rotation. So, the problem of the conventional sample analysis chip that the wax (oil) in a liquid state is stored in the wax chamber leaks during the rotation of the sample analysis chip and mixed with the mixture in the reaction chamber 180 may be prevented.

As described above, the vibration prevention part 400 is configured to be coupled to the second through-hole h of the sample analysis chip 100. To this end, a coupling protrusion 401 to be coupled to the second through-hole h protrudes at the lower portion of the vibration preventing part 400.

The vibration prevention part 400 is coupled to the sample analysis chip 100 after the cartridge 200 is coupled to the sample analysis chip 100. The vibration prevention part 400 has an area sufficient to cover all of the cartridges 200 coupled to the sample analysis chip 100, and thus, prevents an internal solution from leaking to the outside when the sample analysis chip 100 rotates and vibration is generated in the cartridge 200.

### Sample Analysis Method

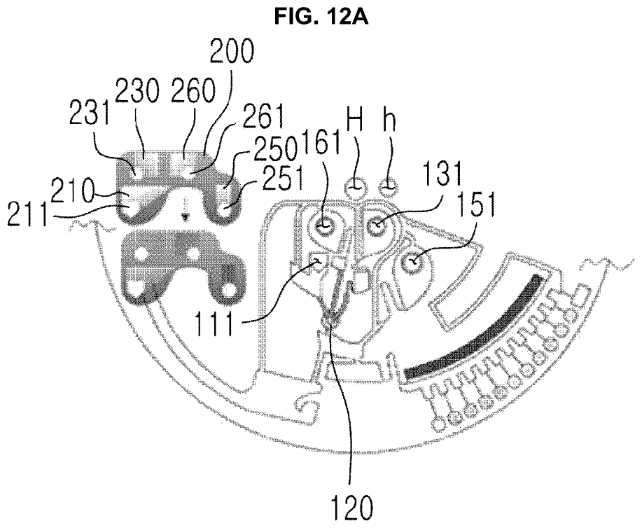

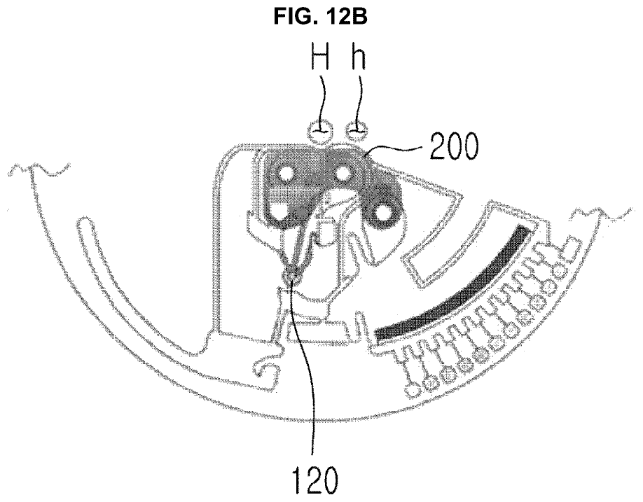

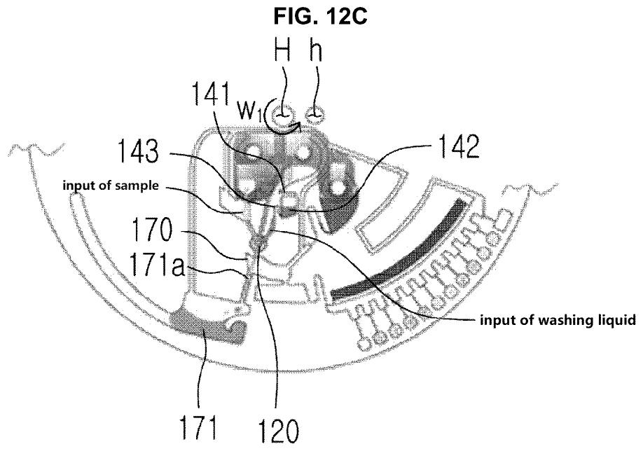

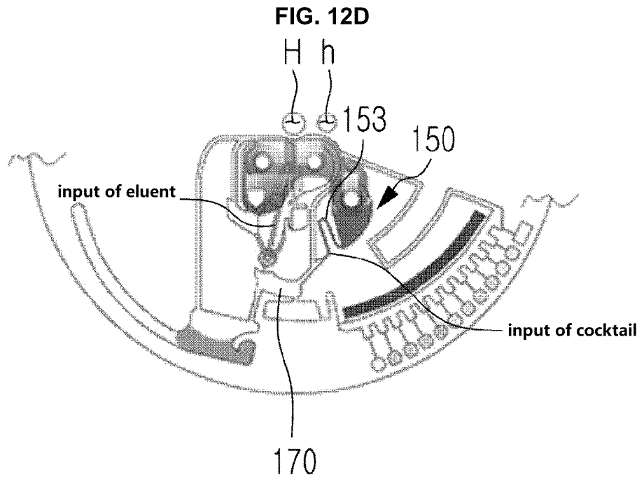

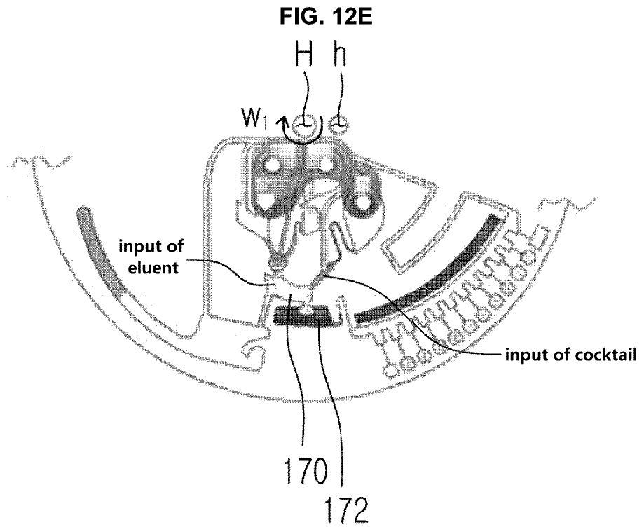

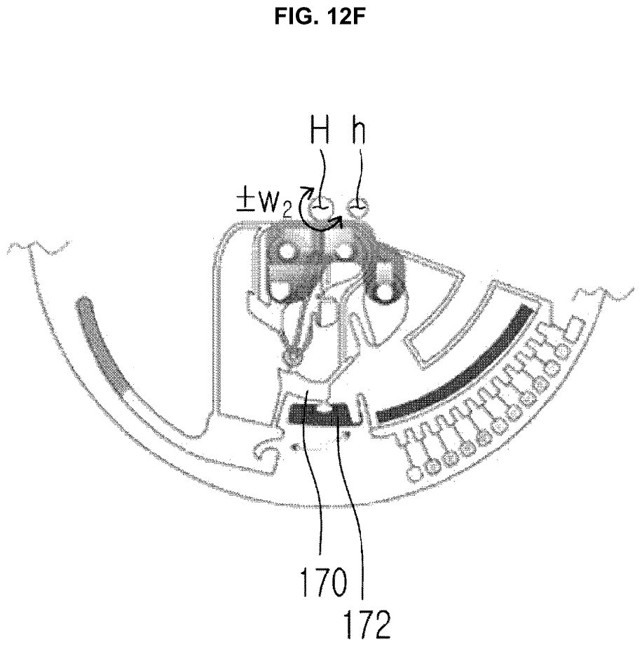

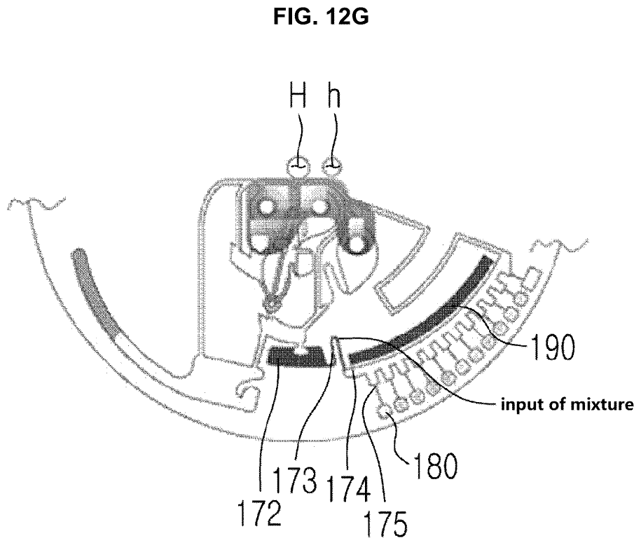

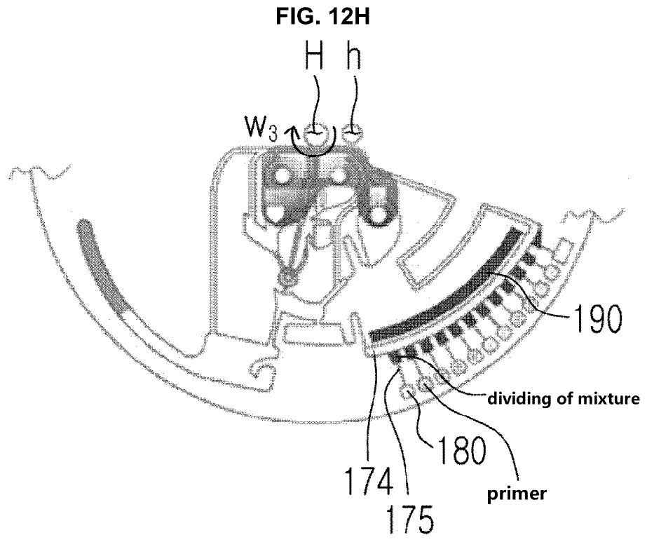

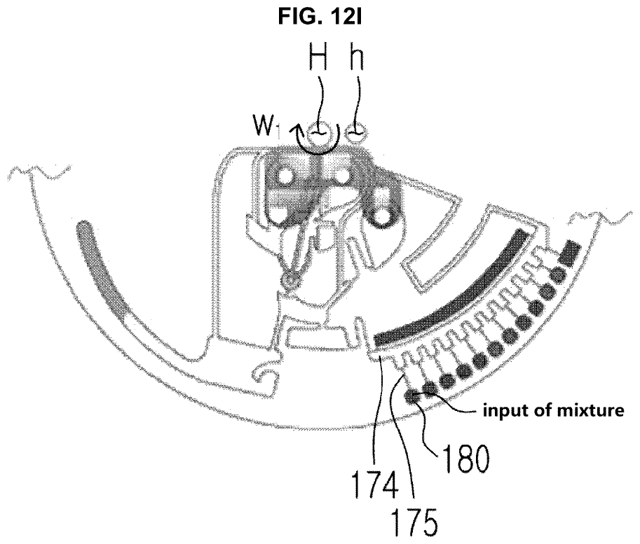

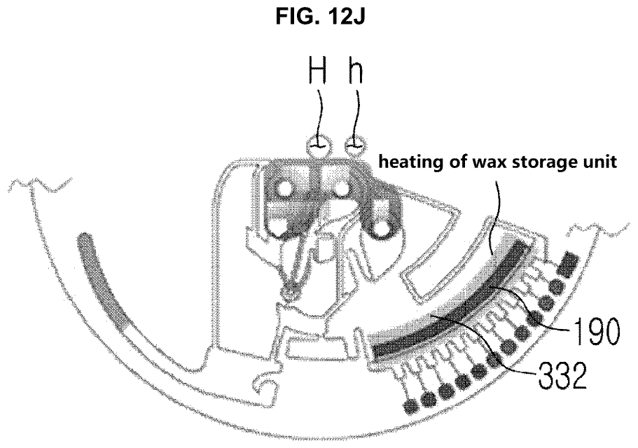

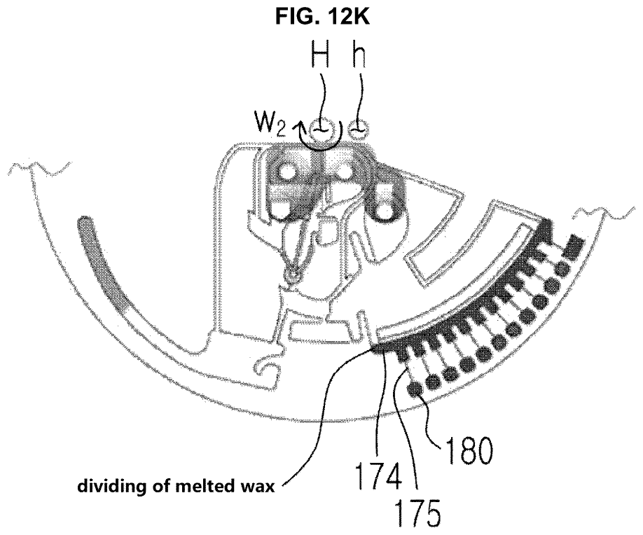

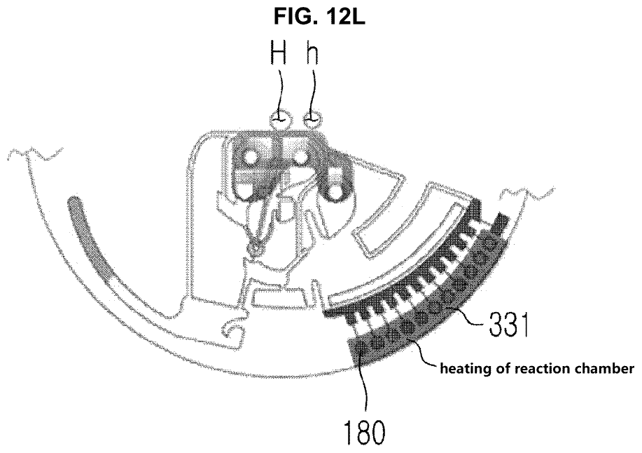

A sample analysis method according to the present invention will be described in detail with reference to FIGS. 12A to 12L.

First, to analyze a sample, each inlet of the cartridge 200 is coupled to the sample storage unit 110, the washing liquid storage unit 130, the cocktail storage unit 150, and the eluent storage unit 160 of the sample analysis chip 100 (FIG. 12B). Accordingly, the solution stored in each solution storage unit of the cartridge 200 is injected into the storage unit of the sample analysis chip 100, and the sample is injected into the sample storage unit 110.

Next, the sample analysis chip 100 is rotated in a first direction for a predetermined time (for example, 90 seconds) at a first rotation speed W₁ (for example, 5000 rpm). According to the rotation, the sample injected into the sample storage unit 110 passes through the capture passage 120, the material to be analyzed included in the sample is captured by the membrane F, and the remaining material that is not captured is introduced into the waste liquid chamber 171 through the connection chamber 170.

As the sample analysis chip 100 continues to rotate, the washing liquid flowing into the first delay chamber 142 and stored therein is captured by the membrane F while passing through the capture passage 220, but the material that is not the material to be analyzed is washed, and the washed material and the washing liquid pass through the connection chamber 170 to be injected into the waste liquid chamber 171 (FIG. 12C).

Next, when the sample analysis chip 100 stops for a predetermined time (for example, 10 seconds), centrifugal force due to rotation is removed, and thus the cocktail stored in the cocktail storage unit 150 and the eluent stored in the eluent storage unit 160 pass through the cocktail introduction channel 153 and the eluent introduction channel 163, respectively (FIG. 12D) by capillary force.

Next, the sample analysis chip 100 rotates about the first through-hole H in a second direction opposite to the first direction for a predetermined time (for example, 30 seconds) at a first rotation speed W₁. According to the rotation, the cocktail passing through the cocktail introduction channel 153 is injected into the connection chamber 170, the eluent, which has passed through the eluent introduction channel 163, separates the material to be analyzed from the membrane F while passing through the capture passage 120, and the eluate containing the material to be analyzed passes through the connection chamber 170 and is introduced into the collection chamber 172 (FIG. 12E).

Next, the sample analysis chip 100 is repeatedly rotated in the first direction and the second direction for a predetermined time (for example, 10 seconds) at a second rotation speed (W₂) (for example, 2000 rpm). According to the rotation, the eluate containing the material to be analyzed injected into the collection chamber 172 and the cocktail are mixed with each other to generate a mixture (FIG. 12F),

Next, the sample analysis chip 100 stops for a predetermined time (for example, 35 seconds). When and the sample analysis chip 100 stops, centrifugal force due to rotation is removed, and thus the mixture generated in the collection chamber 172 is passed through the mixture introduction channel 173 by capillary force (FIG. 12G).

Next, the sample analysis chip 100 is rotated in the second direction for a predetermined time (for example, 35 seconds) at a third rotation speed (W₃) (for example, 1000 rpm). According to the rotation, the mixture moves by a predetermined length along the circumferential direction, and is injected into an input outlet formed outside the input channel 174 having the aliquoting structure. That is, the mixture is sequentially injected into the input outlet of the input channel 174 along the circumferential direction from the point at which the collection chamber 172 and the input channel 174 are connected. Further, the excess mixture that is not injected into the input outlet of the input channel 174 is injected into the excess mixture storage chamber 176 (FIG. 12H).

Next, the sample analysis chip 100 is rotated in the second direction for a predetermined time (for example, 10 seconds) at a first rotation speed (W₁) (for example, 5000 rpm). According to the rotation, the mixture that was injected into each input outlet of the input channel 174 passes through the connecting channel 175 and into each reaction chamber 180 (FIG. 12I).

Next, the sample analysis chip 100 is stopped for a predetermined time (for example, 180 seconds), and the wax storage unit 190 is heated, and thus the wax stored in the wax storage unit 190 is melted (FIG. 12J).

Next, the sample analysis chip 100 is rotated in the second direction for a predetermined time (for example, 30 seconds) at a second rotation speed (for example, 2000 rpm), According to the rotation, the melted wax is fed into the input channel 174 through the wax introduction channel 191. As the melted wax is fed into the input channel 174, the evaporation of the mixture introduced into the reaction chamber 180 is not made (FIG. 12K).

Next, the reaction chamber 180 is heated or cooled according to the temperature of the predetermined reaction cycle. For example, the reaction chamber 180 may be heated or cooled to conform to a temperature suitable for the PCR cycle, thereby amplifying the material to be analyzed introduced into the reaction chamber 180 (FIG. 12L).

### Analysis Apparatus for Sample Analysis Chip

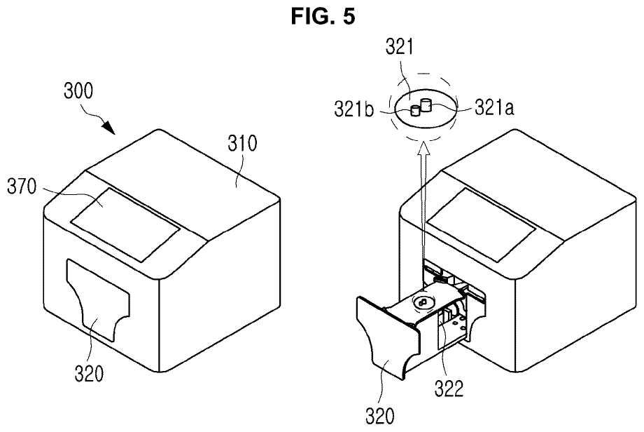

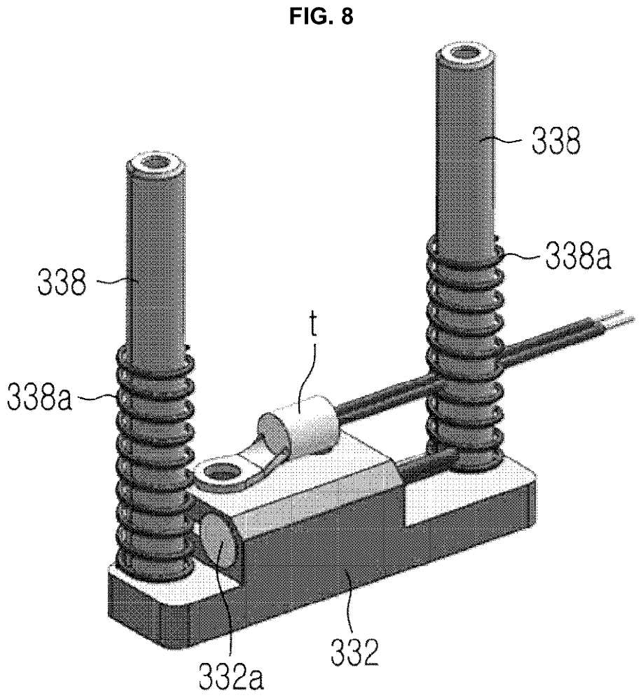

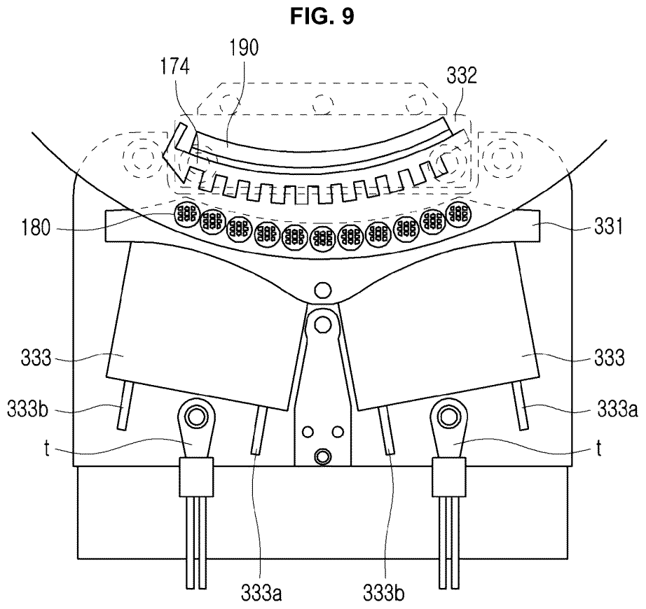

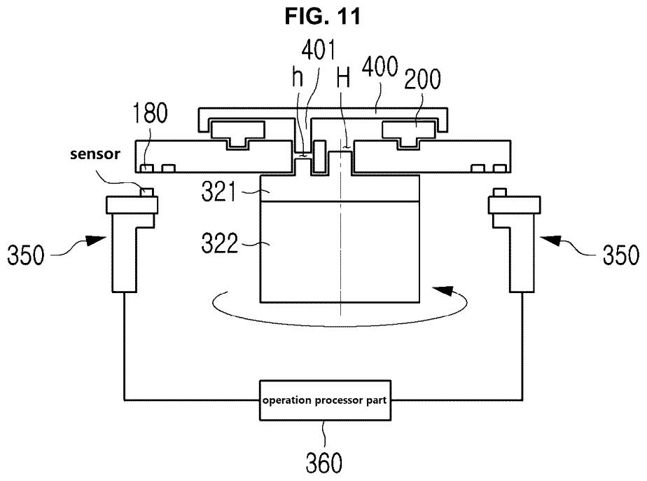

The chip analysis apparatus 300 of the sample analysis chip 100 according to the present invention will be described in more detail with reference to FIGS. 5 to 11.

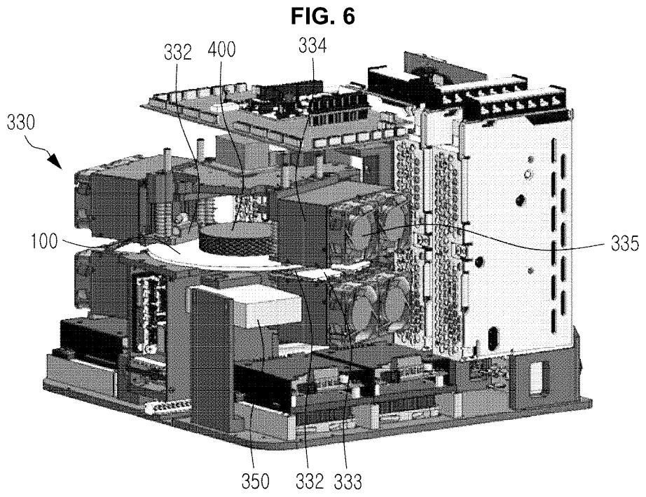

Referring to FIGS. 5 and 6, the chip analysis apparatus 300 according to the present invention includes a housing 310, a chip installation part 320, a heating part 330, a driving part 340, a sensor part 350, an operation processing part 360, and an output part 370.

The housing 310 forms the exterior of the analysis apparatus 300, and the chip installation part 320, the heating part 330, the driving part 340, the sensor part 350, the operation processing part 360, and the output part 370 are installed in the inner space of the housing 310.

The chip installation part 320 is configured such that the sample analysis chip 100 is seated. Specifically, the chip installation part 320 may be accommodated in the housing 310 and may be exposed to the outside for installation of the sample analysis chip 100. That is, the chip installation part 320 is movable in a direction away from or closer to the housing 310 (movable horizontally or vertically with respect to the ground), and a driving part for a chip installation part (not shown) for moving the chip installation part 320 can be installed inside the housing 310. The driving part for the chip installation part (not shown) may be, for example, a step motor, and the chip installation part may be moved between a position where the chip installation part 320 is accommodated in the housing 310 or is exposed to the outside through the control of the step motor.

The chip installation part 320 includes a seating tray 321 on which the sample analysis chip 100 is seated.

A motor 322 for rotating the sample analysis chip 100 is installed in the seating tray 321. Protrusions 321a and 321b coupled to the first through-hole H and the second through-hole h of the sample analysis chip 100 are formed on the upper surface of the seating tray 321, and a motor 322 for rotating the seating tray 321 is installed under the seating tray 321.

Accordingly, as the motor 322 rotates, the sample analysis chip 100 rotates together with the seating tray 321.

The heating part 330 is configured to perform heating of the sample analysis chip 100. Specifically, the heating part 330 includes a first heater 331, a second heater 332, a Peltier element 333, a heat sink 334, a fan 335, a body 336, a first installation column 337, and a second installation column 338.

The first heater 331 serves to heat the reaction chamber 180 of the sample analysis chip 100. The reaction chamber 180 stores a mixture of an eluate containing a material to be analyzed and a cocktail. Generally, the amount of the sample injected into the sample storage unit 110 is a very small amount, and thus the amount of the material to be analyzed included in the sample is a very small amount. Accordingly, in order to detect whether the analysis target material is present in the reaction chamber 180, it is necessary to amplify the analysis target material. In each reaction chamber 180, different primers for amplifying the material to be analyzed are already introduced, and a so-called gene amplification process is performed using the primer and the material to be analyzed.

The gene amplification process may be, for example, a polymerase chain reaction (PCR), and may generally include a denaturation step for separating DNA having a double helix structure into a single strand by heating at a temperature of about 95° C., an annealing step for binding a primer and a single strand of DNA by heating at a temperature of about 56° C., and an extension step that the polymerase extends the strand from the primer by heating at a temperature of about 72° C. In addition, the gene amplification process may be a reverse transcription-polymerase chain reaction (RT-PCR) or an isothermal amplification reaction and is not particularly limited as long as it is a reaction for amplifying a gene to be analyzed.

That is, the first heater 331 serves to amplify the material to be analyzed in the reaction chamber 180 while being sequentially heated to a temperature required for the polymerase chain reaction.

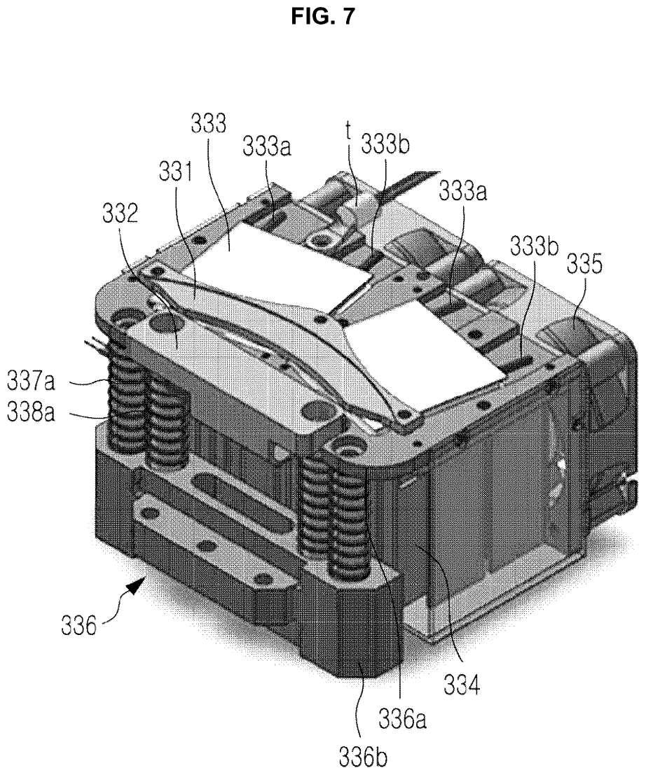

Although the first heater 331 may be directly heated, the first heater 331 may be indirectly heated/cooled by the conduction of heat from the Peltier element 333 to the first heater 331 by heating/cooling of the Peltier element 333 connected to the first heater 331 as shown in FIG. 7. The Peltier element 333 is a device using a Peltier effect, and is a device in which a surface contacting a semiconductor may be heated or cooled according to the intensity and direction of a current flowing through the Peltier element 333. Since the Peltier element is a well-known element, a detailed description thereof will be omitted. The surface of the Peltier element 333 contacting the first heater 331 may be heated or cooled according to the intensity and direction of the current flowing through the Peltier element 333, thereby heating/cooling the first heater 331. That is, the temperature of the first heater 331 may be controlled by varying the intensity and direction of the current flowing through the first wire 333a and the second wire 333b connected to the Peltier element 333.

The first heater 331 may have a curved shape so as to heat alt of the plurality of reaction chambers 180 extending in the circumferential direction in the sample analysis chip 100. That is, the first heater 331 may also have a shape extending along the circumferential direction of the sample analysis chip 100. As shown in FIG. 7, only the reaction chamber 180 including an arc shape may be selectively heated/cooled.

The second heater 332 serves to heat the wax chamber 190 of the sample analysis chip 100. Since the wax stored in the wax chamber 190 is in a solid state, it is necessary to melt the wax into a liquid state. As the second heater 332 is heated, the wax of the wax chamber 190 is melted and introduced into the input channel 174, and is divided into the input outlet of the input channel 174 according to the rotation of the sample analysis chip 100. When the wax is melted, the state of the wax is changed to a liquid state to prevent evaporation of the mixture injected into the reaction chamber 180.

The first heater 331 and the second heater 332 are spaced apart from each other in the heating part 330. This is to prevent heating of portion other than the portion to be heated by the respective heating of the first heater 331 and the second heater 332. The first heater 331 is configured to heat the reaction chamber 180, and the second heater 332 is configured to heat the wax chamber 190. If one heater is heated and affects another heater, inaccurate results may be caused. So, the first heater 331 and the second heater 332 are installed spaced apart from each other. That is, the first heater 331 may be configured to cover all of the reaction chambers 180 provided in the sample analysis chip 100, and the second heater 332 may be configured to cover all the wax chamber 190 provided in the sample analysis chip 100. it is preferable that the separation distance is longer than at least the length of the connection channel 175. As shown in FIG. 7, the second heater 332 may have a rectangular plate shape. Unlike the first heater 331, the second heater 332 does not require precise temperature control. Thus, the second heater 332 may not be connected to the Peltier element 333, and the second heater 332 itself may be heated by heating of other separate heating element 332a to heat the wax chamber 190.

The temperature sensor t may be installed in both the first heater 331 and the second heater 332, and the heating temperature of each heater may be monitored in real time by using information output from the temperature sensor t.

The heating part 330 includes a heat sink 334 and a fan 335 to dissipate heat from the first heater 331, the second heater 332, and the Peltier element 333. As shown in FIG. 7, the heat sink 334 may be installed under the first heater 331, the second heater 332, and the Peltier element 333, and the fan 335 may be installed on one side of the heat sink 334 to quickly discharge heat from the heat sink 334 to the outside.

The body 336 is configured such that the first heater 331, the second heater 332, and the Peltier element 333 are installed and forms the exterior of the heating part. The body 336 includes an upper body 336a in which a first heater 331, a second heater 332, and a Peltier element 333 are installed, and a lower body 336b disposed to face the upper body 336a and connected to a driving part 340 to be described later.

The upper body 336a and the lower body 336b may be connected to each other by a first installation column 337 and a second installation column 338. Springs 337a and 338a are installed outside the respective installation columns 337 and 338. When the driving part 340 operates, the heating part 330 approaches the sample analysis chip 100 and the first heater 331 or the second heater 332 comes into contact with the sample analysis chip 100. In this state, when the driving part 340 applies more force to the heating part 330 toward the direction of the sample analysis chip 100, the springs 337a and 338a are contracted (i.e., the upper body and the lower body are brought closer together) while the first heater 331 and the second heater 332 may be completely attached to the sample analysis chip 100. Accordingly, heat generated in each heater may be fully applied to the sample analysis chip 100.

The driving part 340 is connected to the heating part 330 to move the heating part 330. As shown in FIG. 1, one sample analysis chip 100 may include two unit process parts 100a, and as shown in FIG. 6, one chip analysis apparatus 300 may include four heating parts 330.

The two heating parts 330 per unit process part 100a may be vertically aligned with each other. That is, the unit process part 100a is centered and the heating parts 330 face each other.

After the sample is put into the sample storage unit 110, heating by the heating part 330 is not required until the mixture is injected into the reaction chamber 180 according to the sample analysis method described above. When the mixture is injected into the reaction chamber 180, the melting process of the wax stored in the wax storage unit 190 and the gene amplification process of the reaction chamber 180 are required. In this case, the driving part 340 moves the heating part 330 so that the heating part 330 contacts the sample analysis chip 100.

That is, the driving part 340 moves the heating part 330 such that the heating part 330 moves downward or upward closer to the sample analysis chip 100, and the heating part 330 moves downward or upward away from the sample analysis chip 100. In the chip analysis apparatus 300 according to the present invention, two driving parts 340 are provided, so that the one driving part 340 moves the heating part 330 positioned at the upper side, and the other driving part 340 moves the heating part 330 positioned at the lower side.

The sensor part 350 may be installed above or below the sample analysis chip 100 and configured to detect fluorescence generated in the reaction chamber 180. As the material to be analyzed injected into the reaction chamber 180 undergoes an amplification process, fluorescence intensity increases. When light of a specific wavelength is applied to each reaction chamber 180, the fluorescent material of the reaction chamber 180 absorbs the light of the specific wavelength to become excited, and emits light of a different wavelength while returning to the ground state. That is, the sensor part 350 includes an irradiation unit for emitting light of a specific wavelength to the reaction chamber 180 and a detector for detecting light of a specific wavelength generated by the reaction chamber 180. The sensor part 350 may determine whether the sample includes a material to be analyzed and whether the sample is infected with a disease on the basis of the presence or absence of light of a specific wavelength detected by the detector and the intensity thereof.

As a measurement method of the sensor part 350, point by point method and moving scanning method can be used. In the point by point method, the sensor part 350 is aligned up and down relative to each reaction chamber 180, and whenever the sensor part 350 is aligned, the sensor part 350 irradiates light of a specific wavelength and detects light of a specific wavelength in the reaction chamber 180. In the moving scanning method, while the sample analysis chip 100 rotates, the sensor part 350 continuously irradiates light of a specific wavelength and detects light of a specific wavelength in the reaction chamber 180.

In addition, as an example embodiment, the reaction chamber 180 is configured to accommodate a material generating strong fluorescence in the reaction chamber 180 closest to the collection chamber 172. If the sensor part 350 detects the fluorescence, other reaction chambers can be positioned relative to the corresponding reaction chamber. in other words, since the reaction chamber 180 in which strong fluorescence intensity is detected is predetermined, the position of the reaction chamber 180 can be determined based on the reference reaction chamber 180. The operation processor part 360, which will be described later, can calculate in which reaction chamber fluorescence was detected, which intensity of fluorescence was detected in which reaction chamber 180, through the above-described method.

The operation processor part 360 is configured to determine whether the material to be analyzed is included in the sample and whether the sample is infected with a disease based on the information detected by the detector of the sensor part 350.

For example, when fluorescence is detected in any reaction chamber 180, a material corresponding to the primer (primers severs to perform a gene amplification reaction by specifically binding to the material to be analyzed) may be determined to be included in the sample. This is because the fact that fluorescence of a predetermined intensity was detected means that the gene amplification reaction was performed by the reaction of the material to be analyzed in the sample with the primer stored in the reaction chamber 180.

In addition, when a fluorescence of a predetermined intensity or more is detected in any reaction chamber 180, it may be determined that the disease caused by the analysis target material corresponding to the primer accommodated in the reaction chamber 180 has been infected. Since there is a disease group that cannot be regarded as being infected with a disease simply because the sample contains the analysis target material, it can be determined that the disease caused by the analysis target material has been infected only when fluorescence of a predetermined intensity or more is detected.

### Sample Analysis Method by Analysis Apparatus

First, the chip installation part 320 protrudes out of the housing 310 by driving the driving part of the chip installation part (not shown). The first through-hole H and the second through-hole h of the sample analysis chip 100 are coupled to the protrusions 321a and 321b of the seating tray 321, so that the sample analysis chip 100 is fixed to the seating tray 32.

Next, the chip installation part 320 is accommodated in the housing 310 by driving the driving part of the chip installation part (not shown).

Next, according to the “Sample Analysis Method” described above, the motor 322 of the seating part 321 rotates, and the mixture of the eluate containing the material to be analyzed and the cocktail are put into the reaction chamber 180.

Next, the sample analysis chip 100 rotates such that the second 332 is aligned up and down relative to the wax chamber 190.

Next, the heating part 330 moves closer to the sample analysis chip 100 by the driving part 340, and finally, the second heater 332 is brought into contact with a portion where the wax chamber 190 is formed.

Next, the second heater 332 is heated and the wax in the solid state stored in the wax chamber 190 is melted.

Next, the heating part 330 moves in a direction away from the sample analysis chip 100 by the driving part 340.

Next, the sample analysis chip 100 is rotated to divide the melted wax (oil) into the input channel 174.

Next, the heating part 330 moves closer to the sample analysis chip 100 by the driving part 340, and finally, the first heater 331 is in contact with a portion where the reaction chamber 180 is formed.

Next, the first heater 331 follows a predetermined temperature cycle, for example, the first heater 331 is heated to a temperature of 95° C.→56° C.→72° C., which is a PCR temperature cycle, to amplify the analysis target material of the reaction chamber 180. Here, the temperature control of the first heater 331 may be performed while varying the direction and intensity of the current supplied to the Peltier element 333 connected to the first heater 331.

Next, the heating part 330 moves in a direction away from the sample analysis chip 100 by the driving part 340. The sample analysis chip 100 rotates, so that the sensor part 350 are aligned up and down relative to the reaction chamber 180.

Next, the sensor part 350 irradiates light of a specific wavelength toward the reaction chamber 180 and detects light (fluorescence) of a specific wavelength emitted from the reaction chamber 180. Here, the optical detection method by the sensor part 350 may use the point by point or moving scanning method described above.

Next, the operation processor part 360 determines whether the analysis target material is present in each reaction chamber 180 or whether the sample is infected with a disease by using the fluorescence intensity detected by e sensor part 350. The information of the different primers stored in each reaction chamber 180 is saved the operation processor part 360 in advance. The operation processor part 360 may determine, for example, the reaction chamber 180 having the reference fluorescence intensity as a reference reaction chamber, and determine other reaction chambers based on the determined reference reaction chamber, thereby determining whether the analysis target material is present or whether the sample is infected with a disease in each reaction chamber.

The heating process of the reaction chamber 180 by the first heater 331 and the light detection process by the sensor part 350 may be alternately performed. For example, after the first heater 331 heats the reaction chamber 180 such that the first heater 331 conforms to one PCR temperature cycle, a light detection process by the sensor part 350 may be performed.

Then a heating process by the first heater 331 and a light detection process by the sensor part 350 may be alternately performed again. Through this, it is possible to measure how the fluorescent intensity of each reaction chamber 180 changes as the PCR cycle proceeds.

Through this, the operation processor part 360 may easily determine whether the sample is infected with a disease. Since the reference infection fluorescence intensity determined according to the number of reference PCR cycles is set for each disease, it is possible to determine whether the sample is infected with a disease by using the fluorescence intensity information detected in the reaction chamber 180 whenever the PCR cycle is performed.

### Verification Experiment 1

An experiment was performed to demonstrate the excellent ability of the sample analysis chip 100 according to the present invention.

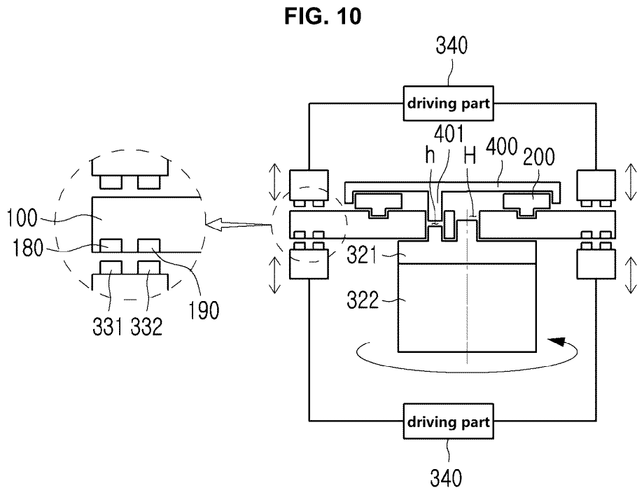

An experiment was performed by injecting a yellow solution into the sample storage unit 110 and the washing liquid storage unit 130, injecting a purple solution into the cocktail storage unit 150 and the eluent storage unit 160, and repeatedly rotating and stopping the sample analysis chip 100 through the process according to FIG. 10.

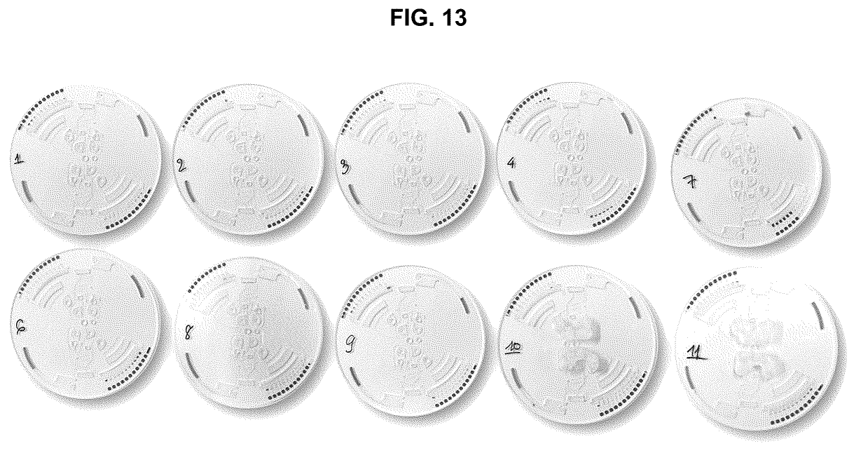

As a result of the experiment, only a purple solution was injected into the reaction chamber 180, and only a yellow solution was injected into the waste liquid chamber 171. That is, the sample analysis chip 100 according to the present invention was able to confirm that each solution is introduced only into the configuration (waste liquid chamber or reaction chamber) to be targeted. It was confirmed that only the mixture (eluent+analysis target material+cocktail) to be analyzed is introduced into the reaction chamber 180 (see FIG. 13).

### Verification Experiment 2

An experiment was performed to compare the efficiency of capturing materials to be analyzed of the sample analysis chip of the present invention with that of a conventional sample analysis chip.

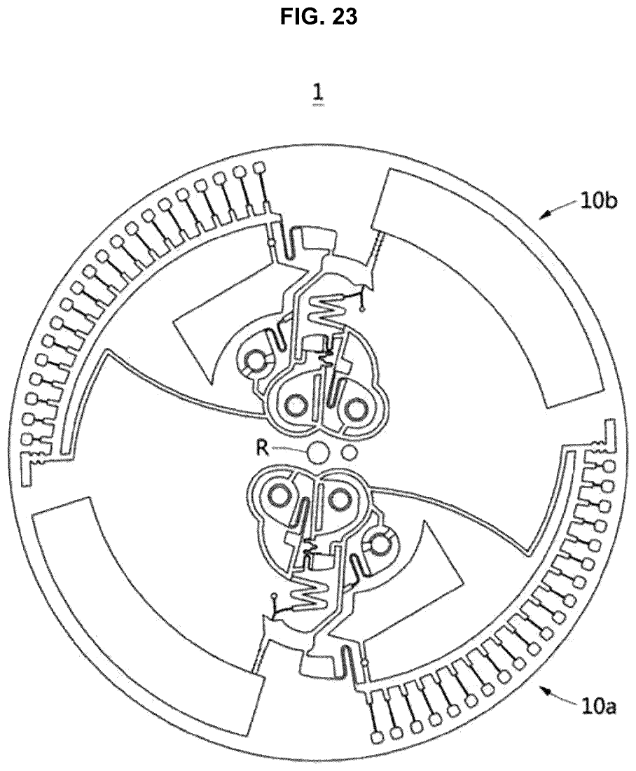

The same amounts of samples were added to the sample analysis chip shown in FIG. 23 and the sample analysis chip 100 according to the present invention, and PCR of the mixture injected into the reaction chamber 180 was performed through the process according to FIG. 12A to FIG. 12L. Also, the same amount of a sample was added to the Qiagen kit, and PCR was performed on the eluate solution.

In addition, the capture efficiency of the sample analysis chip 100 according to the present invention was confirmed (2,3,5, middle layer) while varying the number of layers of silica beads forming the glass filter installed in the capture passage 120 (2 layers, 3 layers, 5 layers)

As a result of the experiment, the conventional sample analysis chip shown in FIG. 23 showed a capture efficiency of about 82.01% and a capture efficiency of 94.47% in the case of the Qiagen kit, whereas the sample analysis chip 100 according to the present invention achieved a capture efficiency beyond 90%. Especially, the capture efficiency of the present invention was 99.94% when a glass filter consisting of 3 layers of silica beads is applied. Considering that all processes of inputting a sample and inputting a washing liquid must be manually performed in the case of the Qiagen kit, the sample analysis chip 100 according to the present invention has not only convenience, but also high efficiency of capturing a material to be analyzed (see FIGS. 14 and 15).

### Verification Experiment 3

In order to verify the superiority of the chip analysis apparatus 300 of the sample analysis chip 100 according to the present invention, a verification experiment was performed.

First, a test experiment was performed on whether the temperature of the reaction chamber 180 may be actually controlled according to the desired temperature cycle by comparing the test experiment in which the thickness of the sample analysis chip 100 is constant throughout the radial direction (thick disc) and the test experiment in which the thickness of a portion where the reaction chamber 180 is located is thinner than that of the other portion (thin disc).

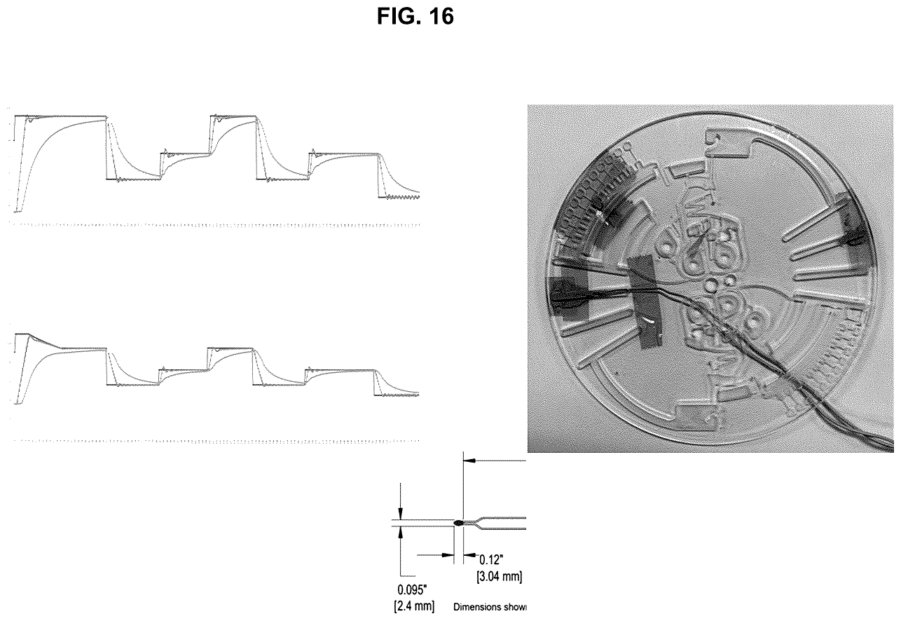

As shown in FIG. 16, the verification experiment was performed by inserting a temperature sensor into the reaction chamber 180 and directly measuring the temperature of the mixture put into the reaction chamber 180.

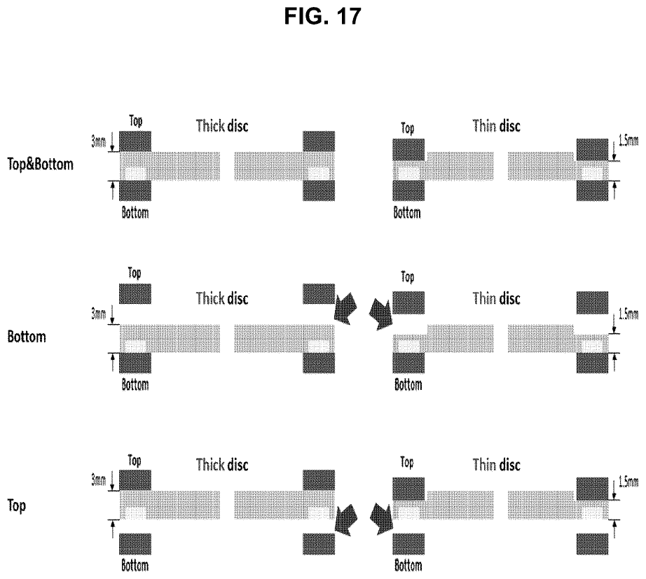

In addition, as shown in FIG. 17, the temperature of the reaction chamber 180 was measured by comparing the test experiment in which both the upper and lower portions of the sample analysis chip 100 are heated in contact with the first heater 331 (Top & Bottom), the test experiment in which only the lower portion of the sample analysis chip 100 are heated in contact with the first heater 331 (Bottom), and the test experiment in which only the upper portion of the sample analysis chip 100 are heated in contact with the first heater 331 (Top).

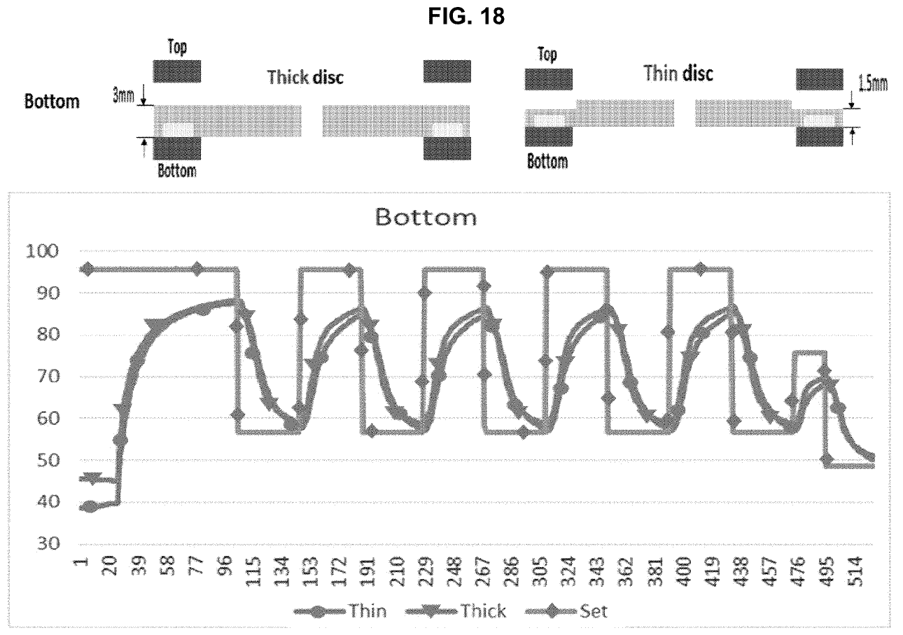

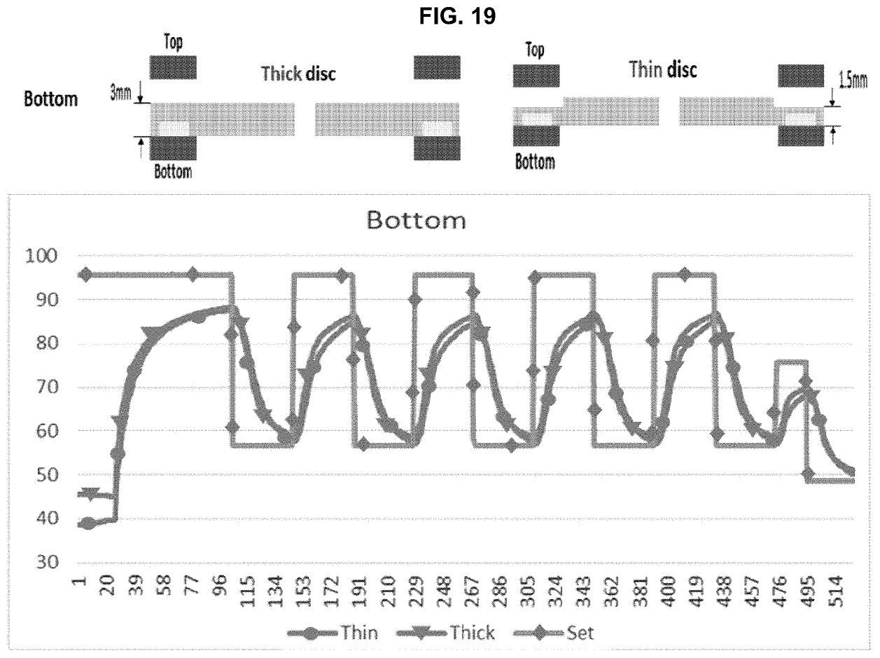

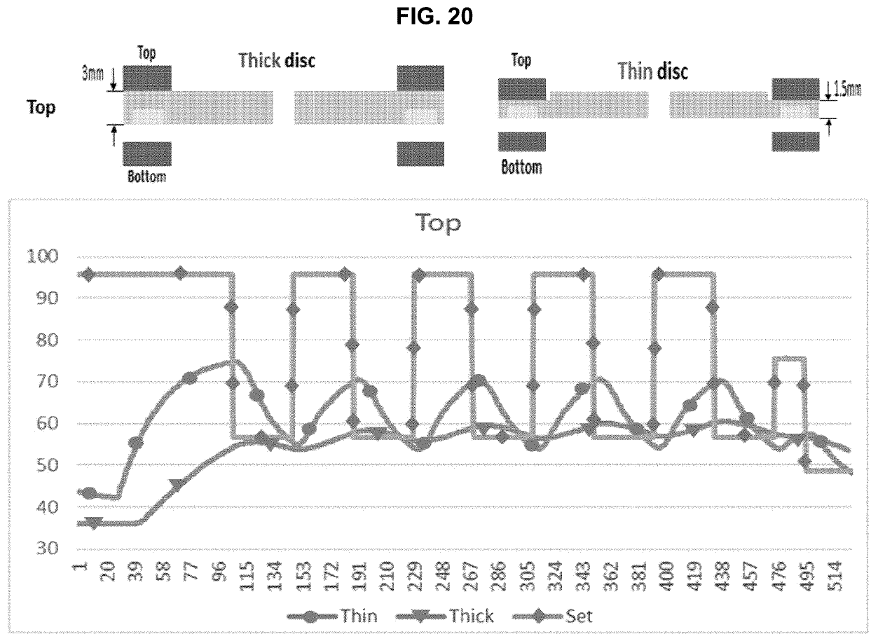

As a result of the experiment, as shown in FIGS. 18 to 20, it was confirmed that the reaction chamber 180 was heated to a temperature similar to the target temperature cycle (PCR temperature cycle) in the test experiment in which both the upper and lower portions of the sample analysis chip 100 was heated (Top & Bottom) rather than the test experiment in which only the upper or lower portion of the sample analysis chip 100 was heated. In addition, although there was no significant difference, it was confirmed that the reaction chamber 180 was heated to a temperature similar to the target temperature cycle for the thin disc rather than the thick disc.

### Verification Experiment 4

A verification experiment was performed on whether each of the plurality of reaction chambers 180 can be identified using the fluorescence intensity detected by the sensor part 350 by using the sample analysis chip 100 according to the present invention.

Materials having different fluorescence intensities are pre-accommodated in each of the reaction chambers (A ref, A1 to A11). While the sample analysis chip 100 is rotated at different speeds, light of a specific wavelength is emitted from the sensor part 350 to the reaction chamber 180 and a fluorescence signal generated therefrom was measured.

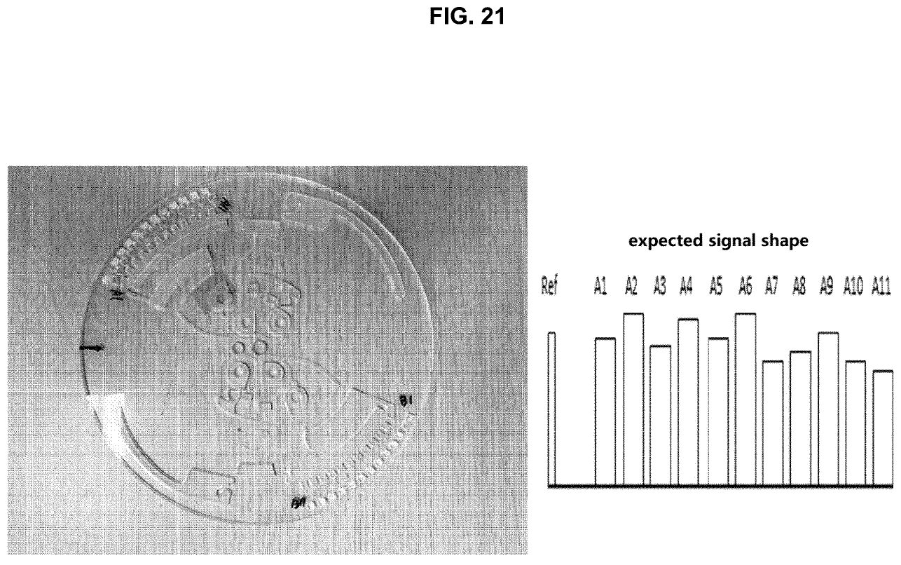

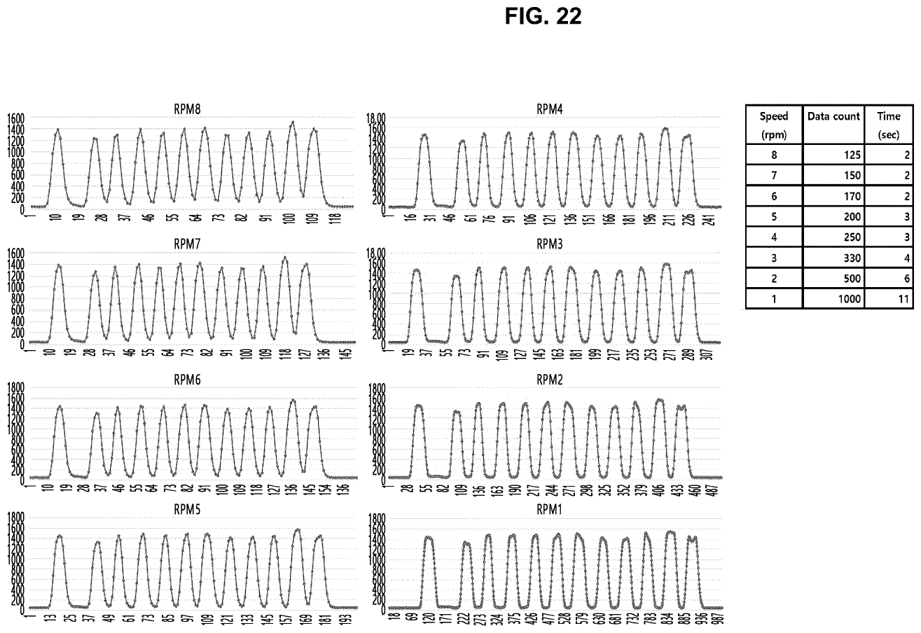

As a result of the experiment, regardless of the rotational speed of the sample analysis chip 100, it was demonstrated that the fluorescence signal generated in each reaction chamber 180 can be detected by the sensor part 350 as the expected signal magnitude. It was also demonstrated that a fluorescence signal generated in the plurality of reaction chambers 180 can be differentiated (see FIGS. 21 and 22).

Although the present invention has been described with reference to the embodiments shown in the drawings so that those skilled in the art can easily understand and reproduce the present invention, this is illustrative only. It will be understood by those skilled in the art that various modifications and other equivalent embodiments are possible from the examples of the present invention. Therefore, the scope of the present invention should be defined by the claims.

(Description of the Drawing Symbols)
100: Sample Analysis Chip
110: Sample Storage Unit
120: Capture Passage
130: Washing Liquid Storage Unit
140: Delay Unit
150: Cocktail Storage Unit
160: Eluent Storage Unit
170: Connecting Chamber
171: Waste Chamber
172: Collection Chamber
174: Input Channel
180: Reaction Chamber
190: Wax Storage Unit
200: Cartridge
300: Chip Analysis Apparatus
310: Housing
320: Chip Installation Part
330: Heating Part
340: Driving Part
350: Sensor Part
360: Operation Processor Part
370: Output Part
400: Vibration Prevention Part

1. A sample analysis chip comprising
a sample storage unit;
a capture passage communicating with the sample storage unit, having a membrane F configured to capture an analysis target material contained in the sample injected into the sample storage unit, and positioned radially outward than the sample storage unit;
a washing liquid storage unit positioned radially inward than the sample storage unit and communicating with the capture passage;
a cocktail storage unit positioned radially inward than the sample storage unit and into which a cocktail for detection of the analysis target material is introduced;
an eluent storage unit positioned radially inward than the sample storage unit, communicating with the capture passage ; and into which an eluent for separating the analysis target material captured in the capture passage is introduced;
a connection chamber positioned radially outward than the capture passage and the cocktail storage unit and communicating with the outlet of the capture passage and the cocktail storage unit;
a collection chamber positioned radially outward than the connection chamber and communicating with the connection chamber, and into which an eluate comprising the analysis target material and the cocktail are introduced when the sample analysis chip is rotated to analyze the sample;
a reaction chamber positioned radially outward than the collection chamber and communicating with the collection chamber; and
a wax storage unit storing a solid wax therein positioned radially inward than the reaction chamber and communicating with the reaction chamber.

2. The sample analysis chip according to claim 1, further comprising:
an input channel positioned radially outward than the collection chamber, positioned radially inward than the reaction chamber, communicating with the collection chamber and the reaction chamber, and into which a mixture of an eluate containing the analysis target material and the cocktail is introduced; and
a connection channel connecting the input channel and the reaction chamber.

3. The sample analysis chip according to claim 2, wherein a plurality of reaction chambers is provided, and the plurality of reaction chambers is formed along the circumferential direction of the sample analysis chip, and the length of the connection channel is shorter as the distance from the collection chamber increases.

4. The sample analysis chip according to claim 1, comprising:
a disk;
a first film layer formed on an upper portion of the disk; and
a second film layer formed on an under portion of the disk, wherein the sample storage unit, the capture passage, the washing liquid storage unit, the cocktail storage unit, the eluent storage unit, the connection chamber, the collection chamber, the reaction chamber, and the wax storage unit are provided in a groove shape in the disk.

5. The sample analysis chip according to claim 4, further comprising:
a first delay chamber provided between the washing liquid storage unit and the capture passage, and
a second delay chamber provided between the eluent storage unit and the capture passage, wherein the sample storage unit, the first delay chamber, and the second delay chamber are provided in a groove shape on an upper portion of the disk,
the connection chamber is provided in a groove shape on an under portion of the disk, and the capture passage is provided in a groove shape extending in an upward direction and a downward direction of the disk so that the sample storage unit, the first delay chamber, and the second delay chamber are communicating with the connection chamber.

6. The sample analysis chip according to claim 5, wherein the membrane F is a glass filter in which a plurality of silica beads is provided in a membrane form, and the glass filter made of three layers of silica beads is installed on the capture passage.

7. The sample analysis chip according to claim 1, wherein injection holes for solution injection are formed in each of the sample storage unit, the washing liquid storage unit, the cocktail storage unit, and the eluent storage unit.

8. The sample: analysis chip according to claim 7, further comprising a cartridge having each inlet and each solution storage unit, wherein the each inlet communicates with the each solution storage unit, and the each inlet is connected to the each injection holes, injecting the sample into the sample storage unit, injecting the washing liquid into the washing liquid storage unit, injecting the cocktail into the cocktail storage unit, and injecting the eluent into the eluent storage unit.

9. The sample analysis chip according to claim 5, further comprising:
an inflow channel; and
a delay channel, wherein the inflow channel is disposed between the washing liquid storage unit and the first delay chamber to allow the washing liquid injected into the washing liquid storage unit to be introduced into the first delay chamber when the sample analysis chip is rotated to analyze the sample,
the inflow channel includes a first passage extending away from the first delay chamber in a first circumferential direction and a second passage extending toward the first delay chamber in a second circumferential direction opposite to the first circumferential direction at an end of the first passage,
the delay channel is disposed between the first delay chamber and the capture passage to allow the washing liquid is discharged into the capture passage when the washing liquid injected into the first delay chamber is greater than a predetermined amount,
the delay channel includes a first delay passage extending radially inward at the outlet of the first delay chamber and a second delay passage extending radially outward than an end of the first delay passage.

10. The sample analysis chip according to claim 1, wherein a thickness of the sample analysis chip where the reaction chamber is provided is thinner than that of other portions of the sample analysis chip.

11. An apparatus in which a sample analysis chip is installed, the sample analysis chip comprises:
a sample storage unit;
a pretreatment unit located radially outward than the sample storage unit and the sample introduced into the sample storage unit is pretreated; and
a reaction chamber located radially outward than the pretreatment unit and into which the sample pretreated by the pretreatment unit is introduced,
the apparatus analyzes a material introduced into the reaction chamber by amplifying the material in the reaction chamber, the apparatus comprises:
a chip installation part on which the sample analysis chip is seated, which includes a motor that rotates the sample analysis chip;
a heating part configured to be movable closer to or away from the reaction chamber in alignment up and down relative to the reaction chamber, and including a first heater for heating the reaction chamber; and
a sensor part in alignment up and down relative to the reaction chamber and capable of detecting fluorescence generated in the reaction chamber.

12. The apparatus according to claim 11, wherein the sample analysis chip further includes a wax storage unit storing a solid wax therein, positioned radially inward than the reaction chamber, and communicating with the reaction chamber,
and the heating part further includes a second heater spaced apart from the first heater in alignment up and down relative to the wax storage unit and heating the wax storage unit.

13. The apparatus according to claim 12, wherein the heating part further includes a Peltier element connected to the first heater.

14. The apparatus according to claim 12, wherein the heating part is installed on an upper side and a lower side of the reaction chamber, respectively.

15. The apparatus according to claim 12, wherein a plurality of reaction chambers is provided, and the plurality of reaction chambers is formed along a circumferential direction of the sample analysis chip, and the first heater extends along the circumferential direction.

16. The apparatus according to claim 15, wherein the area of the first heater is configured to cover at least all of the plurality of reaction chambers, and the area of the second heater is configured to at least cover the wax chamber.

17. The apparatus according to claim 15, wherein a thickness of a portion where the reaction chamber of the sample analysis chip is formed is thinner than that of the other portions.

18. The apparatus according to claim 11, further comprising an operation processor part configured to determine whether a material to be analyzed is present in the sample injected into the sample storage unit or whether the sample is infected with a disease, based on the fluorescence intensity detected by the sensor part.
